(19) United States
(12) Patent Application Publication
(10) Pub. No.: US 2005/0055003 A1
(43) Pub. Date: Mar. 10, 2005

(54) Absorbent tampon comprising a secondary absorbent member attached to the outer surface

(75) Inventors: Dale Francis Bittner, Crosby Township, OH (US); Steven Ray Gilbert, Fairfield, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/656,489

(22) Filed: Sep. 5, 2003

(51) Int. Cl. A61F 13/15; A61F 13/20

(52) U.S. Cl. 604/385.18

(57) ABSTRACT

A catamenial tampon comprising a primary absorbent member and a secondary absorbent member. The primary absorbent member is constructed from an absorbent material compressed to a self-sustaining form. The primary absorbent member has an insertion end, a withdrawal end and outer surface. The secondary absorbent member is fixedly attached to the outer surface of the primary absorbent proximate to the withdrawal end of said primary absorbent member.

### FIELD OF THE INVENTION

This invention relates to absorbent tampons comprising a secondary absorbent member fixed attached to the outer surface.

### BACKGROUND OF THE INVENTION

A wide variety of absorbent catamenial tampons have long been known in the art. While it has been found that these tampons perform their intended function tolerably well, even the best of them do not always re-expand sufficiently, or fast enough, to provide good coverage against leakage. Another common problem with tampons is “bypass” failure that occurs when the menses travels along the length of the vagina without contacting the tampon, i.e., the tampon fails to intercept the flowing menses. During a tampon change, some residual menses may be left near the introitus of the vagina. This may be fluid which was previously absorbed, but which subsequently “squeezed out” of the tampon as it was withdrawn through the sphincter of the vagina. Such residual fluid, particularly if located near the introitus (i.e., in the lower vaginal cavity) may not be effectively absorbed by the replacement tampon. It has been desirable to find a mechanism to absorb bypassed fluid from the lower vaginal cavity. The secondary absorbent member of the present invention provides a mechanism that absorbs bypassed fluid and squeezed out fluid and thus, prevents leakage. In addition, the secondary absorbent member provides a finger grip that consumers may use during the insertion process.

### BACKGROUND OF THE INVENTION

U.S. Pat. No. 6,258,075 issued to Fiona Taylor, et al. relating a TAMPON WITH ENHANCED LEAKAGE PROTECTION.

### SUMMARY OF THE INVENTION

The present invention comprises a catamenial tampon comprising a primary absorbent member and a secondary absorbent member. The primary absorbent member is constructed from an absorbent material compressed to a self-sustaining form. The primary absorbent member has an insertion end, a withdrawal end and outer surface. The secondary absorbent member is fixedly attached to the outer surface of the primary absorbent member proximate to the withdrawal end of the primary absorbent member.

### BRIEF DESCRIPTION OF THE DRAWINGS

FIG. 1 is a perspective view of a tampon of the present invention.

FIG. 2 is a perspective view of the tampon pledget prior to compression into the tampon of the present invention.

FIG. 3 is a perspective view of the tampon from the withdrawal end after folding and prior to compression into the tampon of the present invention.

### DETAILED DESCRIPTION OF THE INVENTION

As used herein “applicator” refers to a device or implement that facilitates the insertion of a tampon, medicament, treatment device, visualization aid, or other into an external orifice of a mammal, such as the vagina, rectum, ear canal, nasal canal, or throat. Non-limiting specific examples of such include any known hygienically designed applicator that is capable of receiving a tampon may be used for insertion of a tampon, including the so-called telescoping, tube and plunger, and the compact applicators, an applicator for providing medicament to an area for prophylaxis or treatment of disease, a spectroscope containing a microcamera in the tip connected via fiber optics, a speculum of any design, a tongue depressor, a tube for examining the ear canal, a narrow hollow pipe for guiding surgical instruments, and the like.

As used herein, “compression” refers to the process of pressing, squeezing, compacting or otherwise manipulating the size, shape, and/or volume of a material to obtain a tampon having a vaginally insertable shape. The term “compressed” refers to the state of a material or materials subsequent to compression. Conversely, the term “uncompressed” refers to the state of a material or materials prior to compression. The term “compressible” is the ability of a material to undergo compression.

The term “digital tampon” refers to a tampon which is intended to be inserted into the vaginal canal with the user's finger and without the aid of an applicator. Thus, digital tampons are typically visible to the consumer prior to use rather than being housed in an applicator.

The term “folded” as used herein, is the configuration of the tampon pledget that may be incidental to lateral compaction of the absorbent material or may purposely occur prior to a compression step. Such a configuration is readily recognizable, for example, when the absorbent material abruptly changes direction such that one part of the absorbent material bends and lies over another part of the absorbent material.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

The “outer surface” of a tampon refers to the visible surface of the (compressed and/or shaped) tampon prior to use and/or expansion. The outer surface may optionally be aesthetically textured, such as with ribs, spiraling ribs, a mesh pattern, etc. Typically, tampons are constructed from an absorbent material, which has been compressed and/or shaped in any or all of the width direction, the radial direction, and the axial direction, in order to provide a tampon which is of a size and stability to allow insertion within the vagina or other body cavity.

As used herein the terms “pledget” or “tampon pledget” are intended to be interchangeable and refer to a construction of absorbent material prior to the compression and/or shaping of such construction into a tampon as described above. Pledgets may be rolled, folded or otherwise manipulated prior to compression. Tampon pledgets are sometimes referred to as a tampon blank, or a softwind, and the term “pledget” is intended to include such terms as well. In general in this specification, the term “tampon” is used to refer to a finished tampon after the compression and/or shaping process. It will be recognized by those of skill in the art that in some contexts these terms are interchangeable. The different stages of tampon manufacture are described herein with an eye toward providing the greatest possible clarity. Therefore, the terms used are to assist the reader in best understanding the features of the invention and not to introduce limitations in the terms not consistent with the context in which they are used in this specification.

The term “rolled” as used herein, is the configuration of the tampon pledget after winding the absorbent material in a spiral round and round upon itself.

A tampon has a “self-sustaining shape” when a tampon pledget has been compressed and/or shaped such that it assumes a general shape and size, which is vaginally insertable, absent external forces. It will be understood by one of skill in the art that this self-sustaining shape need not, and preferably does not persist during actual use of the tampon. That is, once the tampon is inserted and begins to acquire fluid, the tampon may begin to expand and may lose its self-sustaining form.

As used herein the term “tampon,” refers to any type of absorbent structure that is inserted into the vaginal canal or other body cavities for the absorption of fluid and/or gas therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as rectangular, triangular, trapezoidal, semi-circular, hourglass, serpentine, or other suitable shapes. Tampons have an insertion end, withdrawal end, a length, a width, a longitudinal axis and a radial axis. The tampon's length can be measured from the insertion end to the withdrawal end along the longitudinal axis. A typical compressed tampon for human use is 30-60 mm in length. A tampon may be straight or non-linear in shape, such as curved along the longitudinal axis. A typical compressed tampon is 8-20 mm wide. The width of a tampon, unless otherwise stated in the specification, corresponds to the length across the largest cylindrical cross-section, along the length of the tampon.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

As used herein, “cm” is centimeters, “mm” is millimeters, “g/m²” is grams per square meter.

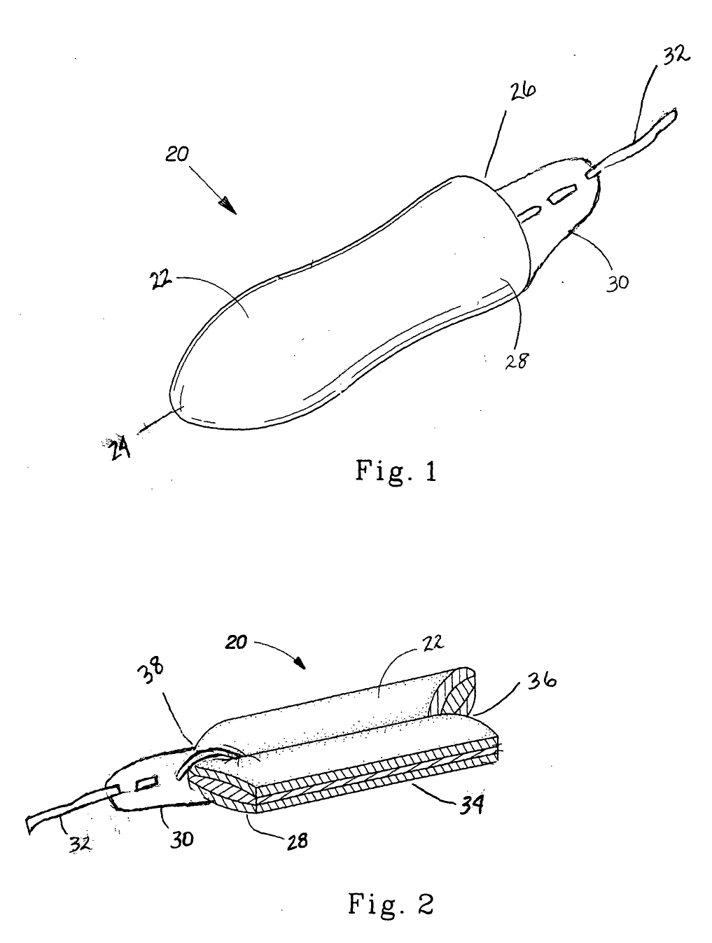

FIG. 1 shows an embodiment of the catamenial tampon 20 of the present invention. The tampon 20 comprises a primary absorbent member 22, and a secondary absorbent member 30. The primary absorbent member 22 is constructed from an absorbent material compressed to a self-sustaining form. The primary absorbent member 22 has an insertion end 24, a withdrawal end 26 and outer surface 28. The secondary absorbent member 30 is attached to the outer surface 28 of the primary absorbent member 22 proximate to the withdrawal end 26 of the primary absorbent member 22. In all embodiments, the secondary absorbent member 30 extends beyond the withdrawal end 26 of the primary absorbent member 22. In the embodiment shown, the tampon 20 comprises a withdrawal member 32 that is joined to the primary absorbent member 22 and extends beyond at least the withdrawal end 26 for removal of the tampon 20.

The tampon 20 of the present invention can be any shape in the art any type of tampon known in the art. The embodiment in FIG. 1 shows a shaped tampon, such as that disclosed in currently pending and commonly assigned, U.S. patent application Ser. No. 10/150,050, filed Mar. 18, 2002, entitled “Substantially Serpentine Shaped Tampon,” to Randall, et al. and currently pending and commonly assigned, U.S. patent application Ser. No. 10/150,055, filed Mar. 18, 2002, entitled “Shaped Tampon,” to Kollowitz, et al.

In some embodiments, both the primary absorbent member 22 and secondary absorbent member 30 may reside entirely within the vaginal cavity of the wearer during use of the tampon 20. This is achieved by the relative closeness of the secondary absorbent member 30 to the withdrawal end 26 of the absorbent material as well of the relative size compared to the overall size of the tampon 20. In some embodiments, only the withdrawal member 32 resides externally to the orifice of the vagina.

FIG. 2 is a perspective view of the tampon pledget 34 prior to compression into the primary absorbent member 22 of a tampon 20 of the present invention. The tampon pledget 34 has a first end 36 and a second end 38. The first end 36 of the tampon pledget 34 corresponds to the insertion end 24 of the primary absorbent member 22 and the second end 38 corresponds to the withdrawal end 26 of the primary absorbent member 22.

Tampon pledgets 34 may be constructed from a wide variety of liquid-absorbing materials commonly used in absorbent articles. Such materials include but are not limited to rayon (such as GALAXY Rayon SARILLE L rayon both available from Acordis Fibers Ltd., of Hollywall, England), cotton, folded tissues, woven materials, nonwoven webs, synthetic and/or natural fibers or sheeting, comminuted wood pulp which is generally referred to as airfelt, or combinations of these materials. Other materials that may be incorporated into the tampon pledget 34 including peat moss, absorbent foams (such as those disclosed in U.S. Pat. No. 3,994,298 issued to DesMarais on Nov. 30, 1976 and U.S. Pat. No. 5,795,921 issued to Dyer, et. al,) capillary channel fibers (such as those disclosed in U.S. Pat. No. 5,356,405 issued to Thompson, et. al on Oct. 18, 1994), high capacity fibers (such as those disclosed in U.S. Pat. No. 4,044,766 issued Kaczmarzk et al. on Aug. 30, 1977), superabsorbent polymers or absorbent gelling materials (such as those disclosed in U.S. Pat. No. 5,830,543 issued to Miyake, et al on Nov. 3, 1998).

The tampon pledget 34 is generally square or rectangular, but other shapes such as trapezoidal, triangular, hemispherical, chevron and hourglass shaped are also acceptable. FIG. 2 shows a tampon pledget 34 that is a chevron shape. A more detailed description of liquid-absorbing materials and pledget shapes and dimensions can be found in currently pending and commonly assigned, U.S. patent Ser. No. 10/039,979, filed Oct. 24, 2001, entitled “Improved Protection and Comfort Tampon,” to Agyapong et al. A typical size for tampon pledget 34 prior to compression may be from about 40 mm to about 100 mm in length and from about 40 mm to about 80 mm in width. In general, the pledget 34 material may be from about 40 mm to about 60 mm in length and from about 50 mm to about 70 mm in width. The typical range for the overall basis weight is from about 150 g/m² to about 800 g/m². The tampon pledget 34 material may be a laminar structure comprised of integral or discrete layers. In other embodiments, the tampon pledget 34 may not have a layered structure at all.

The secondary absorbent member 30 of the present invention may comprise material such as, rayon, cotton, bicomponent fibers, polyethylene, polypropylene, polyester, other suitable natural or synthetic fibers known in the art, and mixtures thereof. The secondary absorbent member 30 may be attached to the second end 38 of the tampon pledget 34, such that after folding and compression, the secondary absorbent member 30 is attached to the outer surface 28 of the primary absorbent member 22, as shown in FIG. 1. In some embodiments, the secondary absorbent member 30 may be fashioned from bottom ply or layer of the tampon pledget 34, such that a portion of the bottom ply extends over the second end 38 of the tampon pledget 34 and thus, the withdrawal end 26 of the resulting primary absorbent member 22. In other embodiments, the secondary absorbent member 30 may extend the full length of the tampon pledget 34 such that the secondary absorbent 30 is attached proximal to the first end 36 of the tampon pledget 34, such that after folding and compression, the secondary absorbent member 30 is attached proximal to the insertion end 24 of the primary absorbent member 22. In other embodiments, the secondary absorbent member 30 is attached proximate to the second end 38 of the tampon pledget 34, such that after folding and compression, the secondary absorbent 30 is attached proximal to the withdrawal end 26 of the primary absorbent member 22. In all embodiments, the secondary absorbent member 30 extends beyond the second end 38 of the tampon pledget 34 and thus, the withdrawal end 26 of the resulting primary absorbent member 22.

The secondary absorbent member 30 may be arranged in a wide variety of shapes and configurations and may be generally cylindrical, spherical, semi-spherical, disc-like, planar, rectangular, “sheet-like,” “skirt-like” in shape. The secondary absorbent member 30 may range in length from about 10 mm to about 40 mm from the second end 38 of the tampon pledget 34 and hence the withdrawal end 26 of the primary absorbent member 22 in length. In some embodiments, the secondary absorbent member 30 may be from about 20 mm to about 25 mm in length. The secondary absorbent member 30 may range from about 6 mm to about 40 mm in width. In some embodiments, the width may range from about 5 mm to about 16 mm. The secondary absorbent member 30 may range in thickness from about 0.5 mm to about 5 mm. In some embodiments, the secondary absorbent member 30 may range in thickness from about 1 mm to about 3 mm.

The secondary absorbent member 30 may be single ply or multiple plies. In some embodiments, the secondary absorbent member 30 may comprise multiple plies of material, each ply having a different capillarity or other absorbent characteristics. In one embodiment with three plies, the outer two layers may have a lower capillarity than the inner ply. In another embodiment, the secondary absorbent member 30 comprises a first ply and a second ply, the first ply has first capillarity and the second ply has a second capillarity and the first capillarity and the second capillarity are different. The secondary absorbent member 30 may be bi-folded, tri-folded or folded any number of times. In some embodiments that are bi-folded, the secondary absorbent member 30 is folded along its length in half. In other embodiments that are bi-folded, the secondary absorbent member 30 is folded along its width in half, so as to form a loop. In some embodiments that are tri-folded, the secondary absorbent member 30 is s-folded along its' length.

The secondary absorbent member 30 may be absorbent and/or hydrophilic. In some embodiments, the secondary absorbent member 30 may have an advancing contact angle greater than the advancing contact angle of the primary absorbent member 22 and/or the withdrawal member 32, such that fluid is preferentially directed toward and absorbed by the primary absorbent member 22. Optionally, the mass of secondary absorbent member 30 may be treated to make it less absorbent than the primary absorbent member 22.

The fluid absorbed and retained by the tampon 20 will ultimately be retained in the primary absorbent member 22. In some embodiments, the secondary absorbent member 30 may be more hydrophilic than the withdrawal member 32. The withdrawal member 32 may be substantially hydrophobic. If the entire withdrawal member 32 not less hydrophilic than the secondary absorbent member 30, at least portions of the withdrawal member 32 are less hydrophilic than the mass of secondary absorbent member 30.

For a more detailed description of hydrophilicity and contact angles see the following publications which are incorporated by reference herein: The American Chemical Society Publication entitled “Contact Angle, Wettability, and Adhesion,” edited by Robert F. Gould, and copyrighted in 1964; and TRI/Princeton Publications, Publication Number 459, entitled “A Microtechnique for Determining Surface Tension,” published in April 1992, and Publication Number 468 entitled, “Determining Contact Angles Within Porous Networks,” published in January, 1993, both edited by Dr. H. G. Heilweil.

The secondary absorbent member 30 may optionally be provided with a mechanism to preferentially direct acquired fluid toward the body of the primary absorbent member 22. Examples of such a driving force are the use of a hydrophilicity gradient as described above. Other mechanisms include a density or capillary gradient, or an osmotic driving force. Capillary channel fibers may optionally be incorporated into the secondary absorbent member 30 in order to provide the driving force for acquired fluid described herein.

The density of material which comprises the secondary absorbent member 30 may in some embodiments be lower than the density of the primary absorbent member 22. In other embodiments, the density of material which comprises the secondary absorbent member 30 may be greater than the density of the primary absorbent member 22. The secondary absorbent member 30 that extends beyond the withdrawal end 26 of the primary absorbent member 22 remains uncompressed or partially compressed during formation of the tampon 20.

The tampon 20 of the present invention may optionally include an overwrap comprising material such as, rayon, cotton, bicomponent fibers, polyethylene, polypropylene, other suitable natural or synthetic fibers known in the art, and mixtures thereof. In some embodiments, the tampon 20 has a nonwoven overwrap comprised of bicomponent fibers that have a polypropylene core surrounded by polyethylene manufactured by Vliesstoffwerke Christian Heinrich Sandler GmbH & Co.KG (Schwarzenbach/Saale, Germany) under the tradename SAS B31812000. In other embodiments, the tampon 20 may comprise a nonwoven overwrap of a hydroentangled blend of 50% rayon, 50% polyester available as BBA 140027 produced by BBA Corporation of South Carolina, U.S. In other embodiments, the overwrap may be 100% polyester. The overwrap may be treated to be hydrophilic, hydrophobic, wicking or non-wicking.

Withdrawal members 32 useful in the present invention may be made of any suitable material known in the prior art and include cotton and rayon. In addition, the withdrawal member 32 can take on other forms such as a ribbon, loop, tab, or the like. The withdrawal member 32 may be integral with the tampon pledget 34 and the resulting primary absorbent member 22. The withdrawal member 32 or regions of the withdrawal member 32 may be treated to be non-absorbent, absorbent or hydrophilic. The withdrawal member 32 may be attached in any suitable manner known in the art including sewing, adhesive attachment, bonding, thermal bonding, or a combination thereof including the method disclosed in currently pending, commonly assigned, U.S. patent application Ser. No. 10/610,075, filed Jun. 30, 2003, entitled “Method and Apparatus for Cord Attachment” to Sargent, et al.

The tampon 20 of the present invention is typically inserted digitally. It may be desirable to provide a finger indent at the withdrawal end 26 of the tampon 20 to aid in insertion, if the tampons 20 are to be digital tampons. A finger indent can be made using a compression rod. An example of finger indents is found in U.S. Pat. No. 6,283,952, filed May 5, 1997, entitled “Shaped Tampon,” issued to Child, et al.

Alternatively, the insertion may be aided through the use of any applicator adapted from the prior art. Prior art applicators of typically a “tube and plunger” type arrangement may be plastic, paper, or other suitable material. Additionally, a “compact” type applicator is also suitable. In some embodiments, where the tampon 20 of the present invention is shaped and provides aesthetic appeal to consumers, it is may be desirable to combine the shaped tampon with an applicator type which enables the user to observe at least a portion or the whole shape of the shaped tampon 20. Two techniques which allow the user to better notice the shape of the tampon 20 are to either make visual observation possible through the use of a translucent or even transparent applicator materials, or to provide a tampon applicator insertion end that better follows and hence better displays the profiled shape of the enclosed shaped tampon than the typical commercial tampon applicators comprising straight-walled cylindrical inserter tubes often made from molded plastic or laminated cardboard tubes. These techniques may be found in currently pending and commonly assigned, U.S. patent application Ser. No. 10/150,055, filed Mar. 18, 2002, entitled “Shaped Tampon,” to Kollowitz, et al.

The tampons 20 of the present invention can optionally employ wrappers which are tightly conforming to the outer surface of the tampon 20 in order to visually show the consumer the tampons packaged therein. Tightly conforming wrappers are particularly useful when the shaped tampons are intended to be used digitally and therefore are not housed in an applicator prior to use. The wrappers should substantially enclose each individual tampon 20 and are intended to be removed prior to insertion and use. “Tightly conforming,” means that there is substantially no visually noticeable void space between the wrapper and the tampon 20. In some embodiments of the present invention some regions of the wrapper material may provide additional functional benefits, such as cord deployment means. Since tampons 20 are typically made by compressing fibrous absorbent material into a self-sustaining shape, the tightly conforming wrapper can optionally be used to act with a certain compressing force on the outer surface of the tampon 20, which will aid maintaining said self-sustaining shape and by counteracting the expansion of the compressed material which otherwise. Such wrappers are discussed in detail currently pending and commonly assigned, U.S. patent application Ser. No. 10/150,055, filed Mar. 18, 2002, entitled “Shaped Tampon,” to Kollowitz.

While several methods of making the tampon 20 of the present invention should be apparent to one of skill in the art in light of the disclosure herein, following is a description of one method of making a tampon 20 of the present invention.

The tampon 20 of the present invention is made by providing the material that comprises the secondary absorbent member 30, the tampon pledget 34, withdrawal member 32, attaching or joining these components, folding the components and compressing. In making the tampon 20 of the present invention, first the material that comprises the secondary absorbent member 30 is provided. Next, the tampon pledget 34 is provided. The material that comprises the secondary absorbent member 30 may be attached to the tampon pledget 34, such that after folding and compression the secondary absorbent member 30 is attached to the outer surface 28 of the primary absorbent member 22, as in FIG. 1. The secondary absorbent member 30 may be attached or joined by any suitable method in the art including sewing, adhesive attachment, bonding, thermal bonding, or a combination thereof. Next, the withdrawal member 32 is attached to the primary absorbent member 22 proximate to the second end 38 of the tampon pledget 34. The withdrawal member 32 may be attached in any suitable manner known in the art including sewing, adhesive attachment, bonding, thermal bonding, or a combination thereof, including the method disclosed in currently pending, commonly assigned, U.S. patent application Ser. No. 10/610,075, filed Jun. 30, 2003, entitled “Method and Apparatus for Cord Attachment” to Sargent, et al. FIG. 2 is a perspective view of the tampon pledget 34 prior to folding and compression.

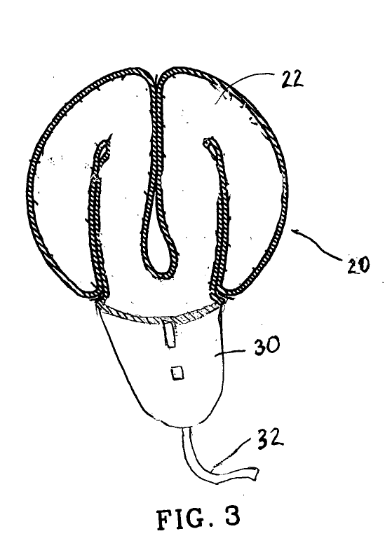

Next, the combination of the tampon pledget 34, secondary absorbent member 30 and withdrawal member 32 are folded such that the tampon pledget 34 is configured in an M-shape, as shown in FIG. 3. To form a tampon ready for use, the tampon pledget 34 is typically compressed and heat conditioned in any suitable conventional manner including the method disclosed in currently pending, commonly assigned, U.S. patent application Ser. No. ______, filed May 12, 2003, entitled “A Process for Producing Stabilized Tampons”, to Prosise, et al. Pressures and temperatures suitable for this purpose are well known in the art. Typically, the tampon pledget 34 is compressed in both the radial and axial direction using any means well known in the art. While a variety of techniques are known and acceptable for these purposes, a modified tampon compressor machine available from Hauni Machines, Richmond, Va., is suitable. Because the secondary absorbent member 30 is smaller in the width dimension than the pledget 34, the radial compression of the pledget 34 will not substantially compress the secondary absorbent member 30. During the axial compression stage, if any, only the pledget 34 is compressed through the use of a suitable push-rod. The secondary absorbent member 30 that extends beyond the withdrawal end 26 of the primary absorbent member 22 remains essentially non-compressed. Optionally, a finger indent can be made using a compression rod. An example of finger indents is found in U.S. Pat. No. 6,283,952, filed May 5, 1997, entitled “Shaped Tampon” issued to Child, et al. In some embodiments, the secondary absorbent member 30 may be attached to the tampon 20 after compression, then no modification of the method of making a conventional compressed absorbent tampon is necessary.

The tampon 20 of the present invention is believed to offer several advantages over prior art tampons. As noted previously, the incorporation of the mass of secondary absorbent member 30 provides absorbency capacity in the lower vaginal vault. This results in a lower disposition of the overall “effective” surfaces of the tampon 20 within the vaginal vault of the wearer. As well, the configuration, shape and size of the secondary absorbent member 30 may provide a finger grip region that allows consumers to properly position the tampon during digital insertion. This is especially the case in embodiments of the tampon 20 having both the secondary absorbent member 30 fixedly attached to the outer surface 28 of the primary absorbent member 22 in combinations with a finger pocket.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the scope of the invention. It is therefore intended to cover in the appended claims all such changes and modifications that are within the scope of this invention.

1. A catamenial tampon comprising:
a primary absorbent member is constructed from an absorbent material compressed to a self-sustaining form, said primary absorbent member having an insertion end, a withdrawal end and an outer surface; and
a secondary absorbent member attached to said outer surface of the primary absorbent member proximate to said withdrawal end of said primary absorbent member.

2. The tampon of claim 1 wherein said secondary absorbent member is sheet-like.

3. The tampon of claim 1 wherein said secondary absorbent member is may range in length from about 10 mm to about 40 mm from said withdrawal end of said primary absorbent member.

4. The tampon of claim 1 wherein said secondary absorbent member is substantially non-compressed.

5. The tampon of claim 1 wherein said secondary absorbent member is a single ply.

6. The tampon of claim 1 wherein said secondary absorbent member comprises a first ply and a second ply.

7. The tampon of claim 6 wherein said first ply has first capillarity and said second ply has a second capillarity; said first capillarity and second capillarity are different.

8. The tampon of claim 1 wherein said secondary absorbent member is bi-folded.

9. The tampon of claim 1 wherein said secondary absorbent member is tri-folded.

10. The tampon of claim 1 further comprising a withdrawal member joined to said primary absorbent member and extending beyond at least said withdrawal end for removal of said tampon.

11. The tampon of claim 1 further comprising a finger indent.

12. A catamenial tampon comprising:
a primary absorbent member is constructed from an absorbent material compressed to a self-sustaining form, said primary absorbent member having an insertion end, a withdrawal end, an outer surface, a finger indent; and
a secondary absorbent member fixedly attached to said outer surface of the primary absorbent member proximate to said withdrawal end of said primary absorbent member.

13. The tampon of claim 11 wherein said secondary absorbent member is sheet-like.

14. The tampon of claim 11 wherein said secondary absorbent member is may range in length from about 10 mm to about 40 mm from said withdrawal end of said primary absorbent member.

15. The tampon of claim 11 wherein said secondary absorbent member is substantially non-compressed.

16. The tampon of claim 11 wherein said secondary absorbent member is a single ply.

17. The tampon of claim 11 wherein said secondary absorbent member is a multiple ply.

18. The tampon of claim 11 wherein said secondary absorbent member is bi-folded.

19. The tampon of claim 11 wherein said secondary absorbent member is tri-folded.

20. The tampon of claim 11 further comprising a withdrawal member joined to said primary absorbent member and extending beyond at least said withdrawal end for removal of said tampon.
